(19) United States
(12) Patent Application Publication
(10) Pub. No.: US 2016/0161585 A1
(43) Pub. Date: Jun. 9, 2016

(54) MAGNETIC RESONANCE APPARATUS AND PROGRAM

(71) Applicant: GE MEDICAL SYSTEMS GLOBAL TECHNOLOGY COMPANY, LLC, Waukesha, WI (US)

(72) Inventors: Yuji WADATE, Hino (JP); Yoshihiro TOMODA, Hino (JP); Kunihiro MIYOSHI, Hino (JP)

(21) Appl. No.: 14/901,520

(22) PCT Filed: Jun. 16, 2014
(86) PCT No.: PCT/US2014/042519

§ 371 Date: Dec. 28, 2015

(30) Foreign Application Priority Data
Jun. 28, 2013 (JP) 2013-136258

(51) Int. Cl. G01R 33/565; G01R 33/483; A61B 5/055; G01R 33/3415

(52) U.S. Cl. CPC G01R 33/56509; G01R 33/3415; G01R 33/483; A61B 5/055

(57) ABSTRACT

To select a channel adapted to detection of the position of a liver. The position “m” of the border between the liver and the lung is obtained from a profile. A sum Sₗᵢᵥₑᵣ of signal intensities in a liver region R1 and a sum S_{lung} of signal intensities in a lung region R2 are calculated. After obtaining the sums Sₗᵢᵥₑᵣ and S_{lung} of the signal intensities, Sₗᵢᵥₑᵣ and S_{lung} are compared to determine whether Sₗᵢᵥₑᵣ is equal to or less than S_{lung}. In the case where Sₗᵢᵥₑᵣ is equal to or less than S_{lung} (Sₗᵢᵥₑᵣ≦S_{lung}), a channel is not selected as a channel used at the time of detecting the position of the edge of the liver. On the other hand, in the case where Sₗᵢᵥₑᵣ is larger than S_{lung} (Sₗᵢᵥₑᵣ>S_{lung}), a channel is selected as a channel used at the time of detecting the position of the edge of the liver.

### CROSS-REFERENCE TO RELATED APPLICATIONS

This is a national stage application under 35 U.S.C. §371(c) of prior filed, co-pending PCT Patent Application No. PCT/US2014/042519, filed on Jun. 16, 2014, which claims priority to Japanese Patent Application No. 2013-136258, filed on Jun. 28, 2013. The aforementioned applications are herein incorporated in their entirety by reference.

### BACKGROUND

The present invention relates to a magnetic resonance apparatus obtaining a navigator signal generated from a navigator region including a body site which moves by using a coil having a plurality of channels and to a program applied to the magnetic resonance apparatus.

Respiration synchronization imaging using a navigator signal is known, refer to Japanese Patent Application No. 2011-193884.

### SUMMARY

In recent years, a multi-channel coil having a plurality of channels is spread, and aspiration synchronization imaging using the multi-channel coil is performed. In the imaging, generally, a navigator region is set in a border position of the liver and the lung and a navigator signal is acquired from the navigator region by the multi-channel coil. On the basis of the navigator signals acquired by the channels in the multi-channel, the position of the edge of the liver is detected. There is, however, a case that depending on the channels, the signal of the lung region is strong. In the case where the signal of the lung region is strong, there is a problem such that the detection precision of the position of the liver is low. Therefore, a technique capable of selecting a channel suitable to detect the position of the liver from the plurality of channels in the case where a channel acquiring the strong signal of the lung region is included in the plurality of channels is demanded.

A first aspect of the present invention relates to a magnetic resonance apparatus obtaining a navigator signal generated from a navigator region including a first body site which moves and a second body site which moves by using a coil having a plurality of channels, including:

scan means executing a first navigator sequence for obtaining a first navigator signal generated from the navigator region;

profile generating means generating a first profile expressing relation between each position in the navigator region and signal intensity for each of the channels on the basis of the first navigator signal received by each of the plurality of channels;

means obtaining a first region corresponding to the first body site in the first profile and a second region corresponding to the second body site in the first profile; and

selecting means selecting a channel used to obtain the position of the first body site from the plurality of channels on the basis of a feature amount of the signal intensity in the first region and a feature amount of the signal intensity in the second region.

A second aspect of the present invention relates to a program applied to a magnetic resonance apparatus executing a first navigator sequence for obtaining a first navigator signal generated from a navigator region including a first body site which moves and a second body site which moves by using a coil having a plurality of channels, the program for making a computer execute:

a profile generating process generating a first profile expressing relation between each position in the navigator region and signal intensity for each of the channels on the basis of the first navigator signal received by each of the plurality of channels;

a process obtaining a first region corresponding to the first body site in the first profile and a second region corresponding to the second body site in the first profile; and

a selecting process selecting a channel used to obtain the position of the first body site from the plurality of channels on the basis of a feature amount of the signal intensity in the first region and a feature amount of the signal intensity in the second region.

A channel is selected on the basis of the feature amount of the signal intensity in the first region and the feature amount of the signal intensity in the second region. Therefore, a channel adapted to obtain the position of the first body site can be selected.

### BRIEF DESCRIPTION OF THE DRAWINGS

FIG. 1 is a schematic diagram illustrating a magnetic resonance apparatus as an embodiment of the present invention.

FIG. 2 is an explanatory diagram of a reception coil 4.

FIG. 3 is a diagram illustrating scans executed in a first mode.

FIG. 4 is a diagram schematically illustrating an imaging region.

FIG. 5 is an explanatory diagram of a sequence executed by a pre-scan A.

FIG. 6 is a diagram illustrating the flow at the time of executing a navigator sequence NAV at time t₁ and detecting the position of the edge of the liver at time t₁.

FIG. 7 is a diagram schematically illustrating profiles F₁ to Fₘ₊ₙ obtained by channels CH₁ to CHₘ₊ₙ of the reception coil 4.

FIG. 8 is an explanatory diagram at the time of determining whether the channel CH₁ is selected or not

FIG. 9 is a diagram illustrating a result of comparison between Sₗᵢᵥₑᵣ and S_{lung}.

FIG. 10 is a diagram illustrating channels CH₂ to CHₘ and CHₘ₊₂ to CHₘ₊ₙ.

FIG. 11 is an explanatory diagram at the time of acquiring the position of the edge of the liver.

FIG. 12 is a diagram illustrating the flow at the time of executing the navigator sequence NAV at time t₂ and detecting the position of the edge of the liver at time t₂.

FIG. 13 is a diagram schematically illustrating profiles F₂ to Fₘ and Fₘ₊₂ to Fₘ₊ₙ generated.

FIG. 14 is a diagram schematically illustrating a composite profile Fc.

FIG. 15 is a diagram illustrating an example of a trigger level TL.

FIG. 16 is an explanatory diagram of a main scan B.

FIG. 17 is an explanatory diagram of an example of a method of selecting a channel by using a template TI.

FIG. 18 is a diagram schematically illustrating a composite profile X obtained by using a method of using the template TI.

### DETAILED DESCRIPTION

Hereinafter, modes for carrying out the invention will be described. The present invention, however, is not limited to the following modes.

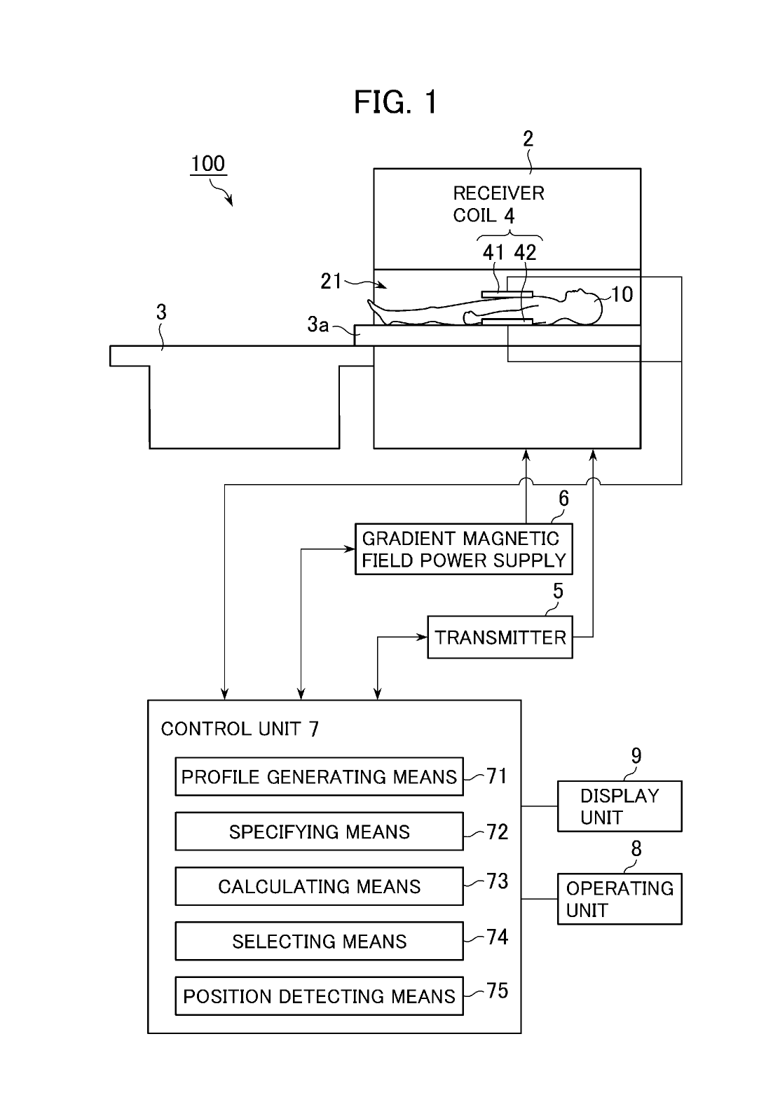

FIG. 1 is a schematic diagram illustrating a magnetic resonance apparatus as an embodiment of the present invention. A magnetic resonance apparatus (hereinbelow, called an “MR apparatus”) 100 has a magnet 2, a table 3, a reception coil 4, and the like.

The magnet 2 has a bore 21 in which a subject 10 is put. The magnet 2 has therein a superconductive coil, a gradient coil, an RF coil, and the like.

The table 3 has a cradle 3a supporting the subject 10. The cradle 3a is configured to be movable in the bore 21. By the cradle 3a, the subject 10 is carried into the bore 21. The reception coil 4 receives a magnetic resonance signal from the subject 10.

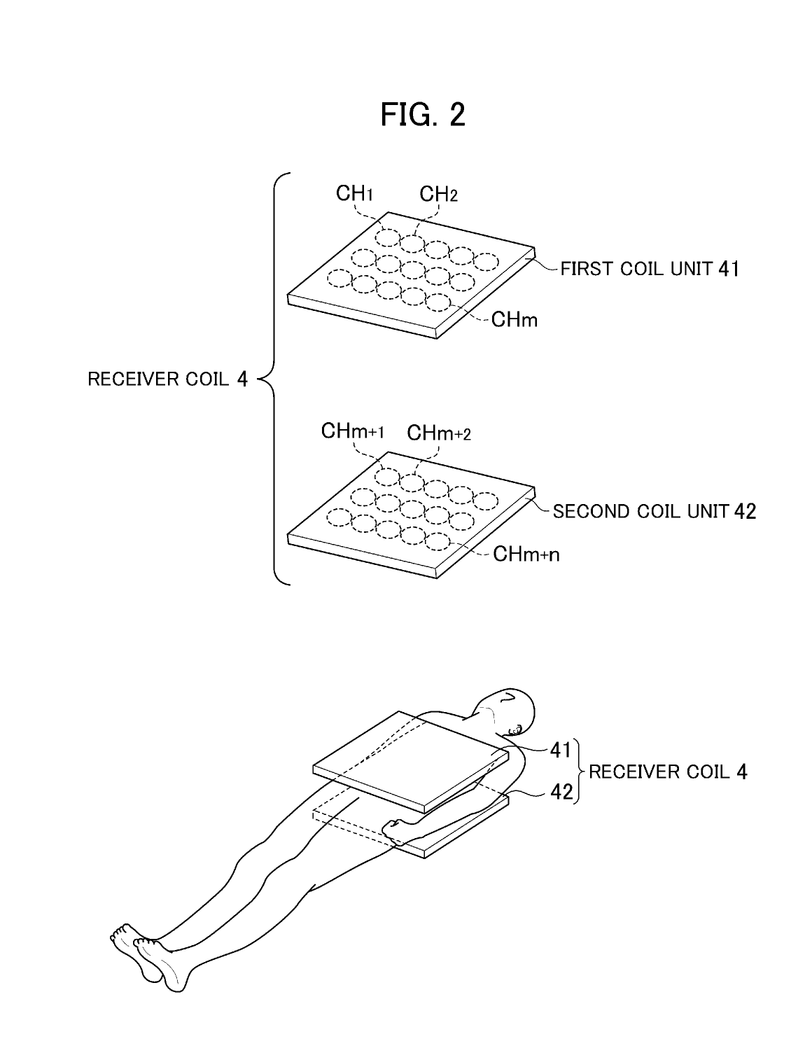

FIG. 2 is an explanatory diagram of the reception coil 4. The reception coil 4 has a first coil unit 41 and a second coil unit 42. The first coil unit 41 has m pieces of channels CH₁ to CHₘ for receiving a magnetic resonance signal from the subject, and the second coil unit 42 has n pieces of channels CHₘ₊₁ to CHₘ₊ₙ for receiving a magnetic resonance signal from the subject. Therefore, in the embodiment, the reception coil 4 is constructed as an (m+n)-channel coil. The first coil unit 41 is disposed on the abdomen side of the subject, and the second coil unit 42 is disposed on the back side of the subject. Referring again to FIG. 1, the description will be continued.

The MR apparatus 100 further has a transmitter 5, a gradient magnetic field power supply 6, a controller 7, an operator 8, a display unit 9, and the like. The transmitter 5 supplies current to the RF coil, and the gradient magnetic field power supply 6 supplies current to the gradient coil. A combination of the magnet 2, the reception coil 4, the transmitter 5, and the gradient magnetic field power supply 6 corresponds to scan means.

The controller 7 controls the operations of the components of the MR apparatus 100 so as to realize various operations of the MR apparatus 100 such as transmission of necessary information to the display unit 9 and reconfiguration of an image on the basis of signals received from the reception coil 4. The controller 7 includes profile generating means 71 to position detecting means 75.

The profile generating means 71 generates a profile expressing the relation between each of positions in the navigator region and signal intensity. Specifying means 72 specifies a region corresponding liver and a region corresponding lung in each profile. Calculating means 73 calculates a sum of signal intensities in the liver region and a sum of signal intensities in the lung region. Selecting means 74 selects a channel adapted to detect the position of the edge of the liver from the channels CH₁ to CHₘ₊ₙ of the reception coil 4 on the basis of the sum of the signal intensities in the liver region and the sum of signal intensities in the lung region. The position detecting means 75 detects the position of the edge of the liver.

The controller 7 is an example of constructing the profile generating means 71 to the position detecting means 75 and functions as those means by executing a predetermined program.

The operator 8 is operated by the operator and enters various information to the controller 7. The display unit 9 displays various information. The MR apparatus 100 is constructed as described above.

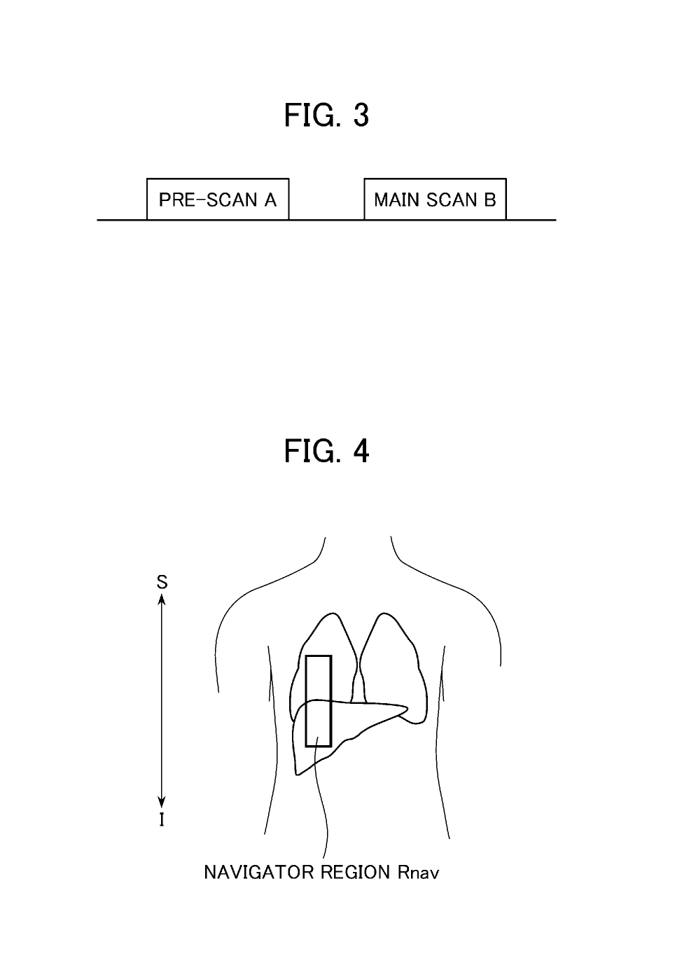

FIG. 3 is a diagram illustrating scans executed in a first mode, and FIG. 4 is a diagram schematically illustrating an imaging region. In the first embodiment, a pre-scan A and a main scan B are executed.

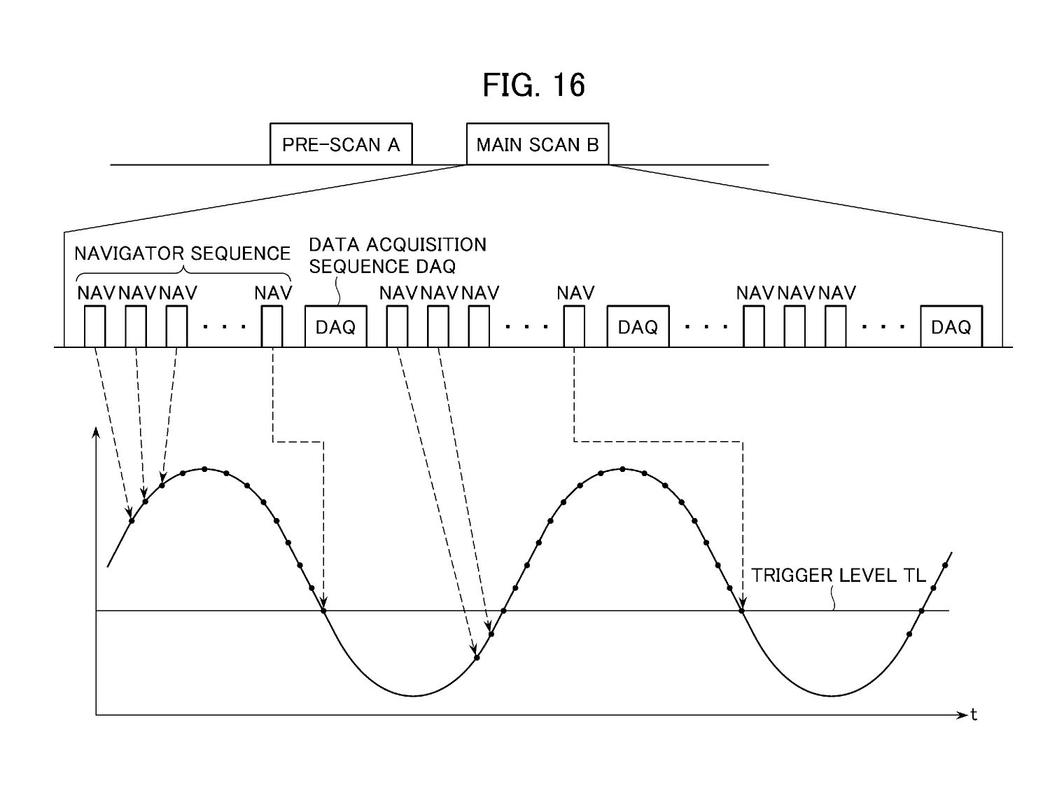

The pre-scan A is a scan executed to determine a trigger level TL (refer to FIG. 16) which will be described later. The trigger level TL will be described later. The main scan B is a scan for imaging the liver. Hereinbelow, the pre-scan A and the main scan B will be described in order.

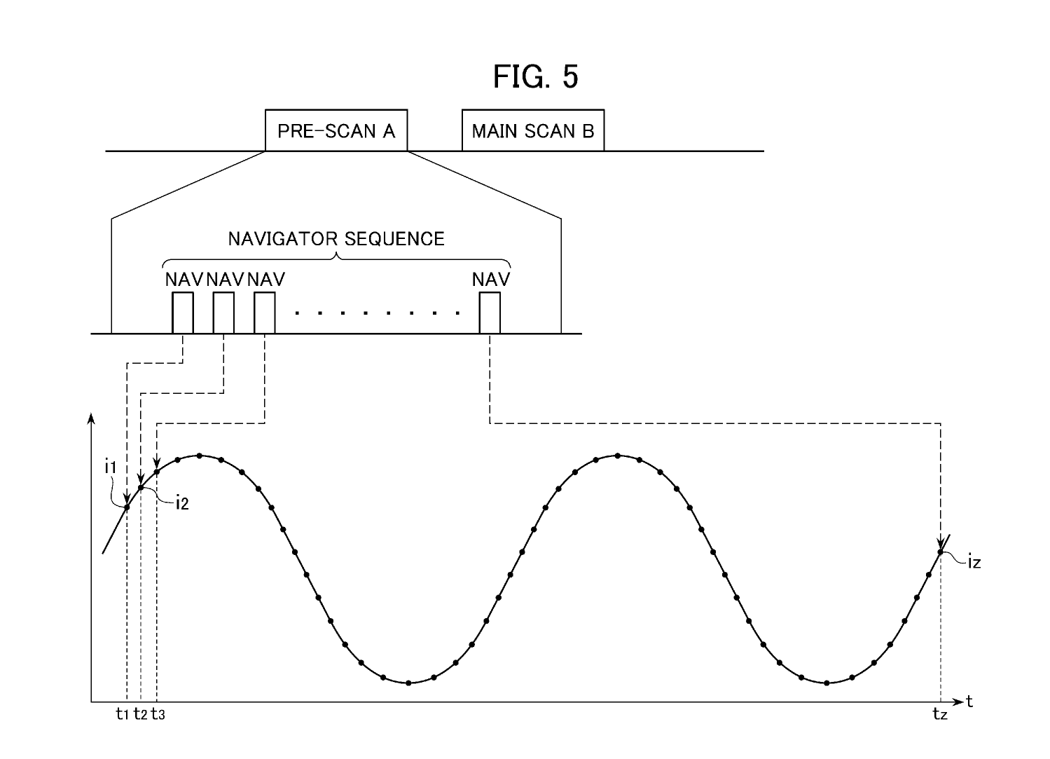

FIG. 5 is an explanatory diagram of a sequence executed by the pre-scan A. In the pre-scan A, a navigator sequence NAV is repeatedly executed. The navigator sequence NAV is a sequence for collecting a navigator signal from a navigator region Rₙₐᵥ.

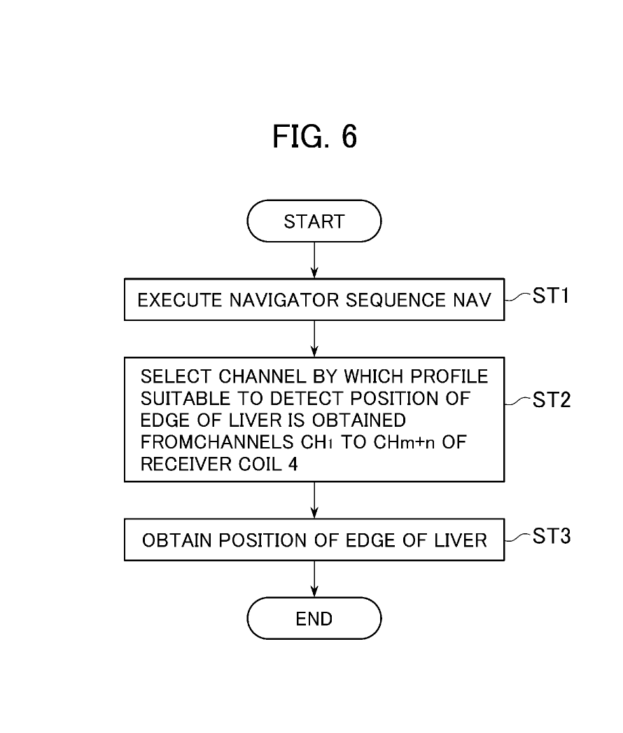

In the pre-scan A, first, the navigator sequence NAV is executed at time t1 to detect the position of the edge of the liver at time t1 (refer to FIG. 6).

FIG. 6 is a diagram illustrating the flow at the time of executing the navigator sequence NAV at time t1 and detecting the position of the edge of the liver at time t1.

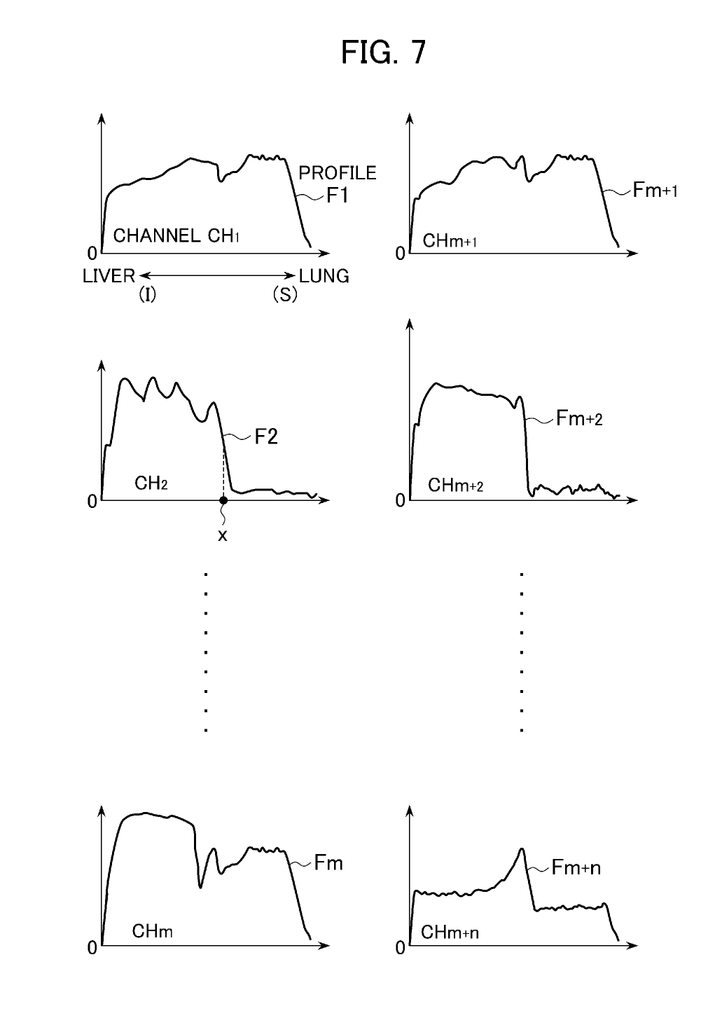

In step ST1, the navigator sequence NAV is executed at time t1. By executing the navigator sequence NAV, the navigator signal is obtained from the navigator region Rₙₐᵥ. The navigator signal is received by each of the channels CH₁ to CHₘ₊ₙ of the reception coil 4. The profile generating means 71 (refer to FIG. 1) converts the navigator signal obtained by each of the channels CH₁ to CHₘ₊ₙ of the reception coil 4 to a profile expressing the relation between each position in the SI direction of the navigator region Rₙₐᵥ and signal intensity. By the operation, a profile is generated for each of the channels of the reception coil 4. FIG. 7 schematically illustrates profiles F₁ to Fₘ₊ₙ obtained by the channels CH₁ to CHₘ₊ₙ of the reception coil 4, respectively. The navigator sequence NAV is designed so that a high signal corresponds to the liver and the low signal corresponds to the lung. Therefore, by detecting the position when the signal values of the profiles F₁ to Fₘ₊₁ change drastically, the position of the edge of the liver at time t1 can be detected. For example, referring to the profile F₂, the signal intensity changes drastically in position x, so that the position x can be therefore considered as the position of the edge of the liver.

There is, however, the case that, depending on the channels of the coil, the signal intensity in the region of the lung in the profile is high. For example, in the profile F₁, the signal intensity in the region of the lung is high. When the signal intensity in the region of the lung is high as described above, the position when the signal intensity changes drastically appears not only in the vicinity of the edge of the liver but also in the region of the lung. It causes erroneous detection of the position of the edge of the liver. Therefore, although (m+n) pieces of the profiles F₁ to Fₘ₊ₙ are obtained by the channels C₁ to CHₘ₊ₙ, it does not mean that a profile suitable to detect the position of the edge of the liver is obtained from all of the channels.

It is consequently necessary to select a channel from which a profile suitable to detect the position of the edge of the liver from the channels CH₁ to CHₘ₊ₙ. To select a channel, the program advances to step ST2.

In step ST2, on the basis of the profiles of the channels, a channel used at the time of detecting the position of the edge of the liver is selected from the channels CH₁ to CHₘ₊ₙ. Hereinafter, a method of selecting a channel in the embodiment will be described.

In the case of selecting a channel, whether or not the channel CH₁ is selected as a channel used at the time of detecting the position of the edge of the liver from the channels CH₁ to CHₘ₊₁ is determined. The determination is performed as follows.

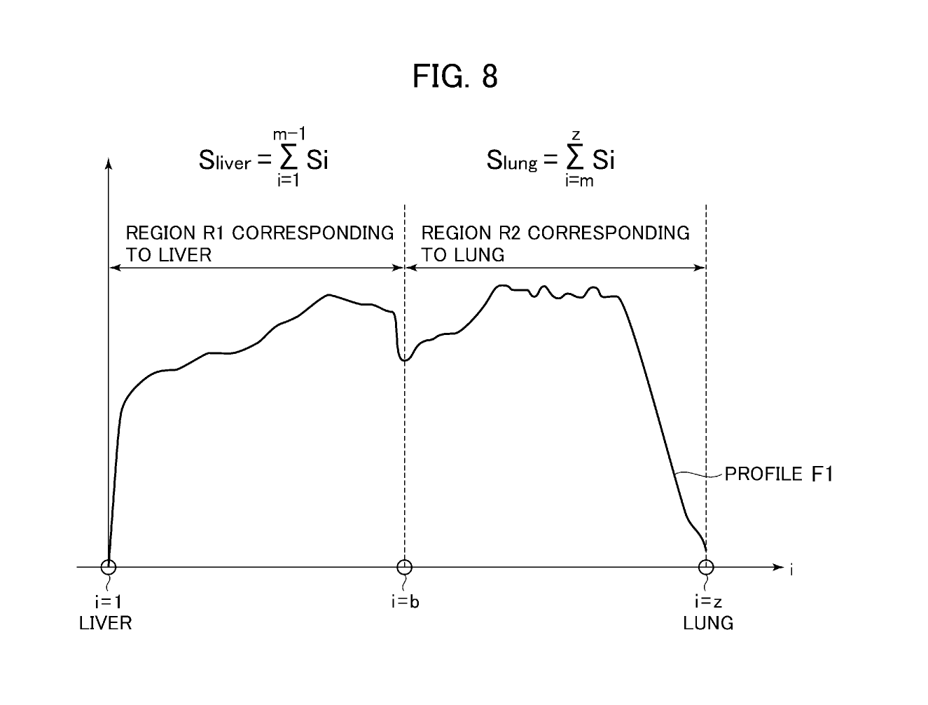

FIG. 8 is an explanatory diagram at the time of determining whether the channel CH₁ is selected or not.

First, the specifying means 72 (refer to FIG. 1) obtains position “b” of the border between the liver and the lung on the basis of the profile F₁ of the channel CH₁. As a method of obtaining the position “b” of the border, various methods are considered. For example, by combining all of the profiles F₁ to Fₘ₊ₙ, a composite profile is obtained. The position where the signal intensity changes drastically is detected from the composite profile, and the detected position can be considered as the position “b” of the border in the profile F₁.

It is sufficient that the position “b” of the border expresses a rough position of the border between the liver and the lung, and it is unnecessary to accurately obtain the position of the border. Therefore, an intermediate position in the SI direction of the navigator region may be set as the position “b” of the border.

The specifying means 72 specifies two regions in the profile F₁ using the position “b” of the border as a reference, that is, a region R₁ corresponding to the liver (hereinbelow, called “liver region”) and a region R₂ corresponding to the lung (hereinbelow, called “lung region”).

Next, the calculating means 73 (refer to FIG. 1) calculates a sum Sₗᵢᵥₑᵣ of signal intensities in the liver region R1 and a sum S_{lung} of signal intensities in the lung region R2. The sums Sₗᵢᵥₑᵣ and S_{lung} of the signal intensities can be obtained by the following equations.

$$\begin{matrix}{S}_{liver} = {\sum }_{i = 1}^{b - 1} {S}_{i} & ( 1 ) \\ {S}_{lung} = {\sum }_{i = b}^{z} {S}_{i} & ( 2 )\end{matrix}$$

where i: position in the SI direction, and Si: signal intensity in the position “i”.

After obtaining the sums Sₗᵢᵥₑᵣ and S_{lung} of the signal intensities, the selecting means 74 (refer to FIG. 1) compares Sₗᵢᵥₑᵣ and S_{lung} and determines whether Sₗᵢᵥₑᵣ is equal to or less than S_{lung}. In the case where Sₗᵢᵥₑᵣ is equal to or less than S_{lung} (Sₗᵢᵥₑᵣ≦S_{lung}), it is considered that the signal intensity in the region of the lung is high, so that the selecting means 74 determines not to select the channel CH₁ as a channel used at the time of detecting the position of the edge of the liver. On the other hand, in the case where Sₗᵢᵥₑᵣ is larger than S_{lung} (Sₗᵢᵥₑᵣ>S_{lung}), it is considered that the signal intensity in the region of the lung is low, so that the selecting means 74 selects the channel CH₁ as a channel used at the time of detecting the position of the edge of the liver.

It is assumed here that Sₗᵢᵥₑᵣ≦S_{lung}. Therefore, the selecting means 74 determines not to select the channel CH₁ as a channel used at the time of detecting the position of the edge of the liver.

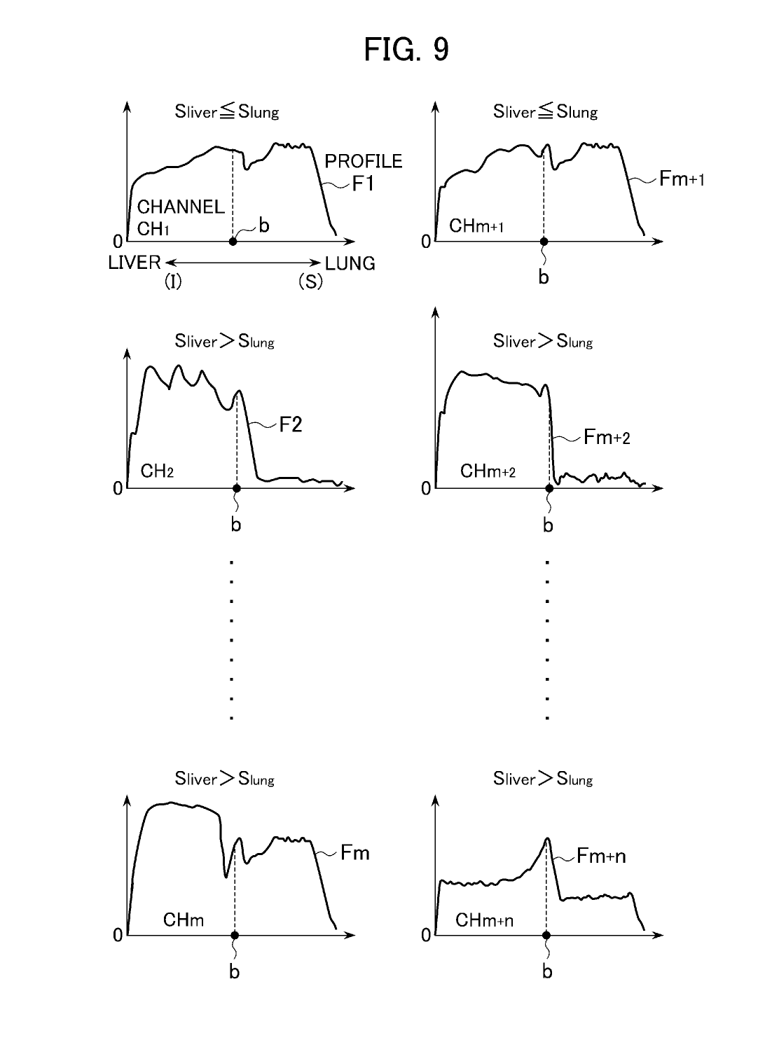

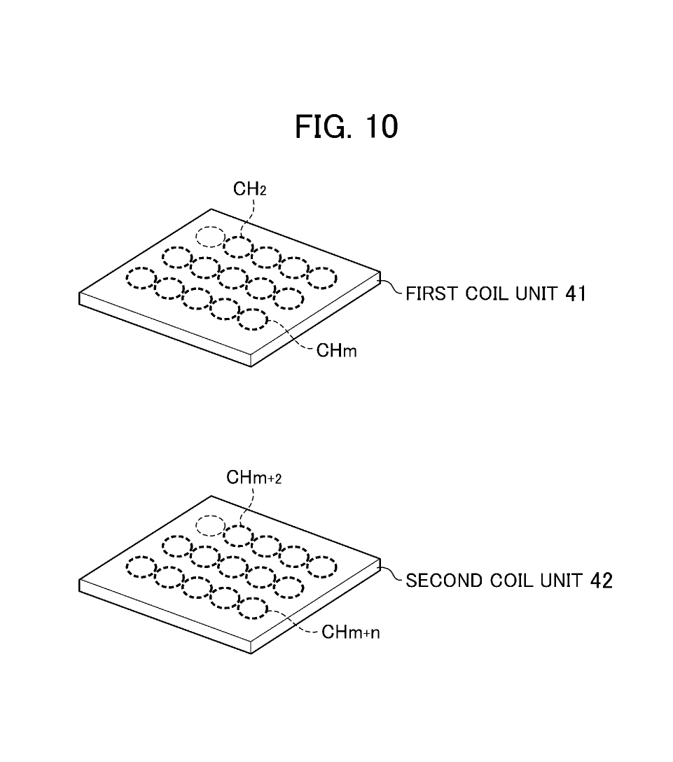

Similarly, the position “b” of the border is set also for the profile F₂ of the channel CH₂ to the profile Fₘ₊ₙ of the channel CHₘ₊ₙ, and the sums Sₗᵢᵥₑᵣ and S_{lung} of the signal intensities are calculated by the equations (1) and (2). Sₗᵢᵥₑᵣ and S_{lung} are compared. In the case where Sₗᵢᵥₑᵣ≦S_{lung}, the selecting means 74 determines not to select the channel as a channel used at the time of detecting the position of the edge of the liver. On the other hand, in the case of Sₗᵢᵥₑᵣ>S_{lung}, the selecting means 74 selects the channel as a channel used at the time of detecting the position of the edge of the liver. FIG. 9 illustrates a result of comparison between Sₗᵢᵥₑᵣ and S_{lung} in each of the profiles F₁ to Fₘ₊ₙ of the channels CH₁ to CHₘ₊ₙ. It is assumed that Sₗᵢᵥₑᵣ≦S_{lung} is satisfied in the profile F₁ of the channel CH₁ and the profile Fₘ₊₁ of the channel CHₘ₊₁, and Sₗᵢᵥₑᵣ>S_{lung} is satisfied in the profiles of the other channel CH₂ to CHₘ and CHₘ₊₂ to CHₘ₊ₙ. Therefore, the selecting means 74 determines not to select the channels CH₁ and CHₘ₊₁ as channels used at the time of detecting the position of the edge of the liver, and to select the other channels CH₂ to CHₘ and the channels CHₘ₊₂ to CHₘ₊ₙ as channels used at the time of detecting the position of the edge of the liver. In FIG. 10, the channels CH₂ to CHₘ and CHₘ₊₂ to CHₘ₊ₙ are indicated by thick broken lines. After selecting the channels, the program advances to step ST3.

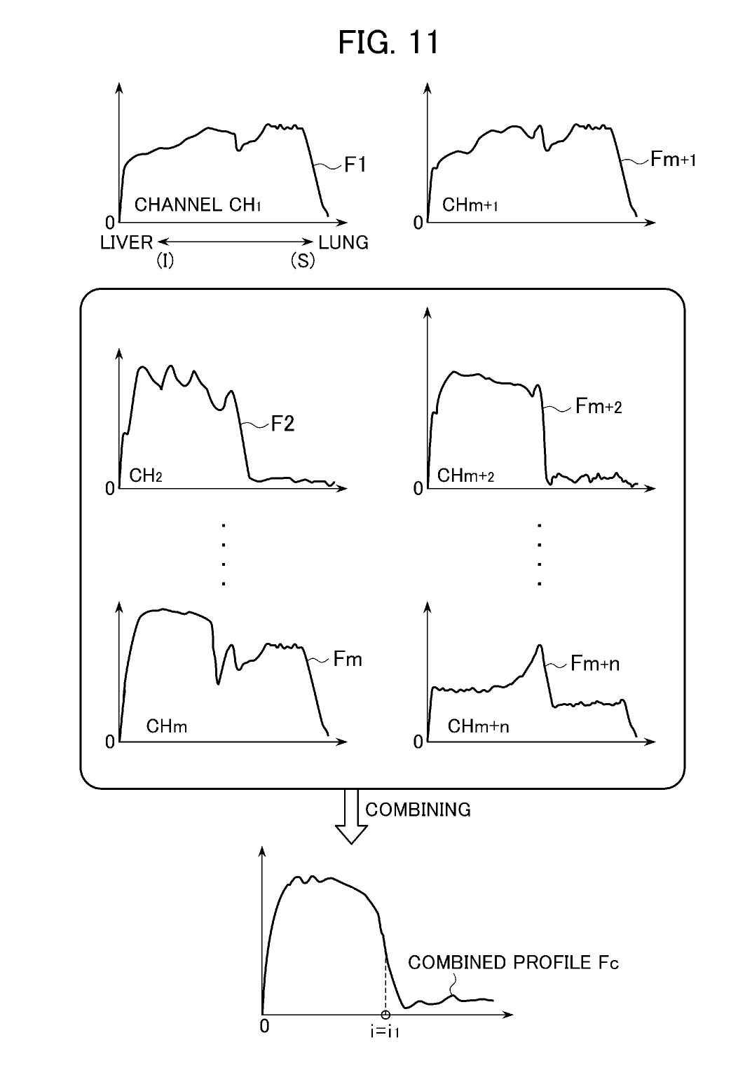

In step ST3, based on the profiles F₂ to Fₘ and Fₘ₊₂ to Fₘ₊ₙ obtained by the channels CH₂ to CHₘ and CHₘ₊₂ to CHₘ₊ₙ, the position of the edge of the liver at time t1 is obtained (refer to FIG. 11).

FIG. 11 is an explanatory diagram at the time of acquiring the position of the edge of the liver. The position detecting means 75 (refer to FIG. 1), first, combines the profiles F₂ to Fₘ and Fₘ₊₂ to Fₘ₊ₙ to obtain a composite profile Fc. In this case, the position detecting means 75 obtains the composite profile Fc by calculating the root mean of the signal intensities of the profiles F₂ to Fₘ and Fₘ₊₂ to Fₘ₊ₙ.

The position detecting means 75 detects the position i=i1 when the signal intensity changes drastically from the composite profile Fc. Consequently, the position i1 (refer to FIG. 5) of the edge of the liver at time t1 can be detected. By combining the profiles F₂ to Fₘ and Fₘ₊₂ to Fₘ₊ₙ, the SN ratio can be increased, so that the detection precision of the position of the edge of the liver can be improved. After obtaining the position i1 of the edge, the flow of FIG. 6 is finished.

After detecting the position p1 of the edge of the liver at time t1, the navigator sequence is executed at the following time t2.

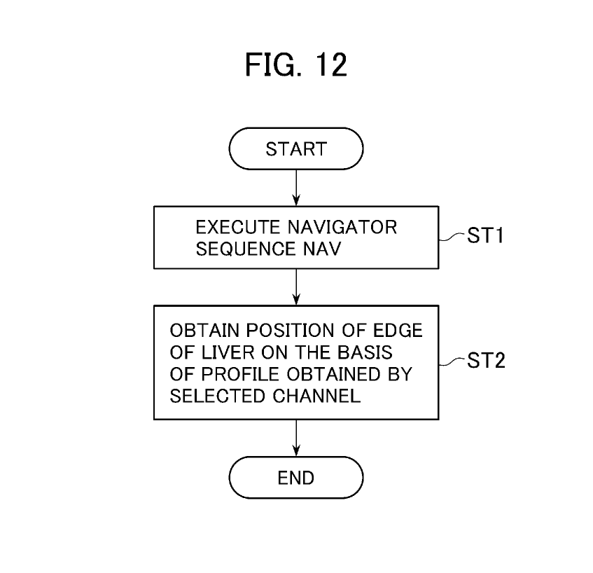

FIG. 12 is a diagram illustrating the flow at the time of executing the navigator sequence NAV at time t2 and detecting the position of the edge of the liver at time t2.

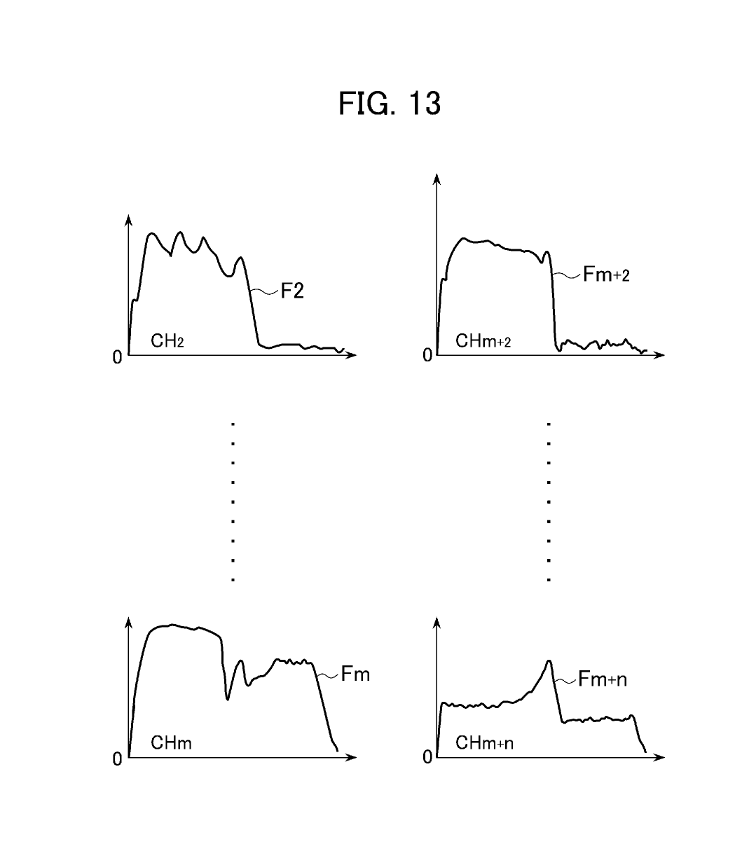

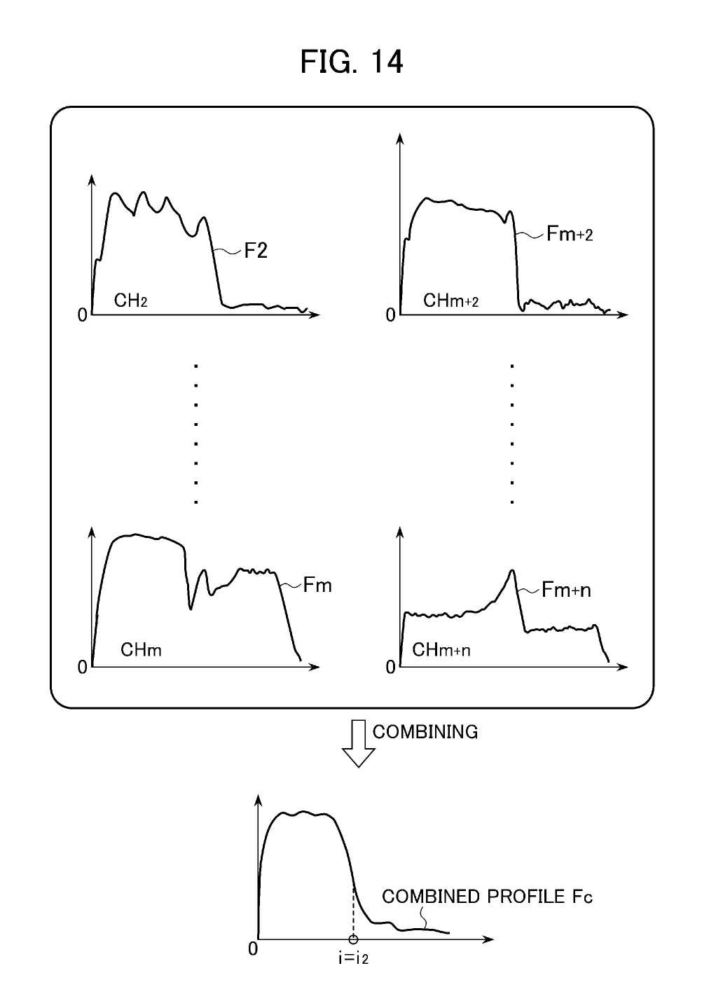

In step ST1, the navigator sequence NAV is executed at time t2. By executing the navigator sequence NAV, the navigator signal is obtained from the navigator region Rₙₐᵥ. The profile generating means 71 converts the navigator signals received by the channels CH₂ to CHₘ and CHₘ₊₂ to CHₘ₊ₙ (refer to FIG. 10) to profiles each expressing the relation between each position in the SI direction of the navigator region Rₙₐᵥ and signal intensity. By the conversion, profiles are generated for the channels CH₂ to CHₘ and CHₘ₊₂ to CHₘ₊ₙ. FIG. 13 schematically illustrates the profiles F₂ to Fₘ and Fₘ₊₂ to Fₘ₊ₙ generated. After obtaining the profiles F₂ to Fₘ and Fₘ₊₂ to Fₘ₊ₙ, the position detecting means 75 calculates the root mean of the signal intensities of the profiles F₂ to Fₘ and Fₘ₊₂ to Fₘ₊ₙ to obtain the composite profile Fc. FIG. 14 schematically illustrates the composite profile Fc.

The position detecting means 75 detects the position i2 when the signal intensity changes drastically from the composite profile Fc. In such a manner, the position i2 (refer to FIG. 5) of the edge of the liver at time t2 can be detected.

Similarly, also at time t3 to tz (refer to FIG. 5), according to the flow shown in FIG. 12, the navigator sequence NAV is executed, and profiles are generated by using the navigator signals received in the selected channels CH₂ to CHₘ and CHₘ₊₂ to CHₘ₊ₙ. The profiles are combined and the position of the edge of the liver is detected from the composite profile.

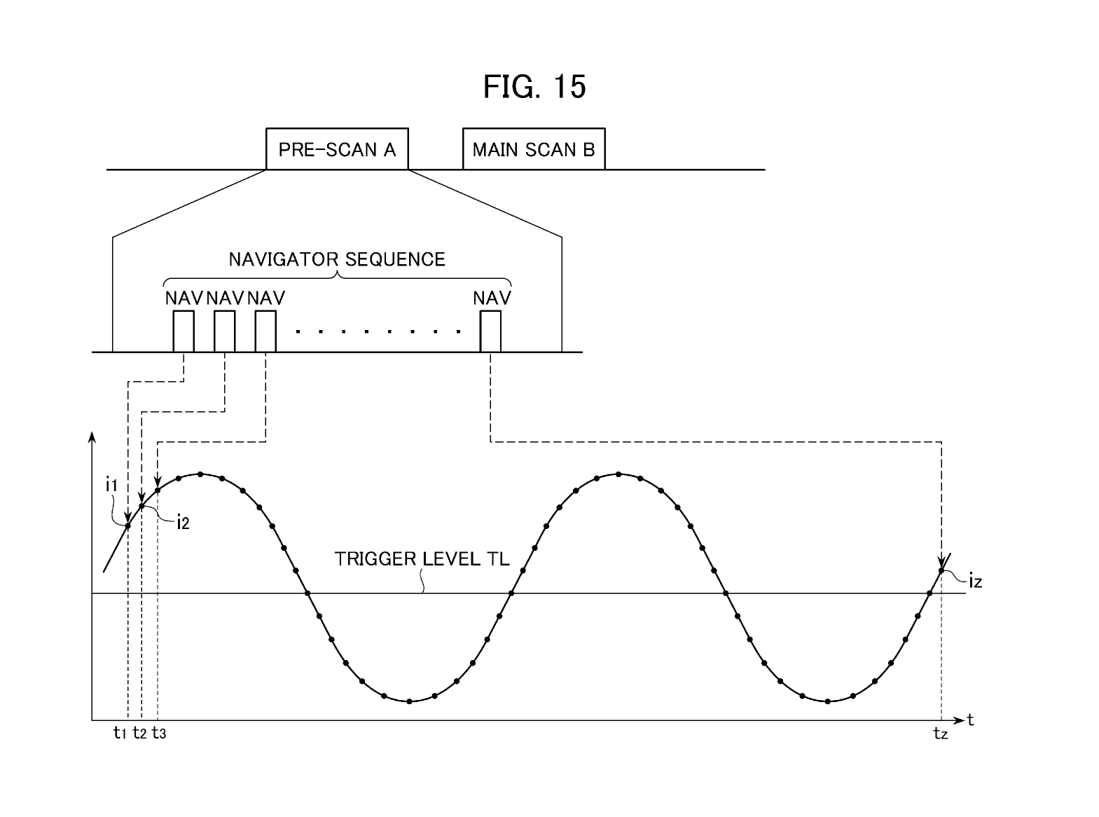

Therefore, as illustrated in FIG. 5, data of the positions i1 to iz of the edge of the liver at time t1 to tz can be obtained. After obtaining the data, on the basis of the data of the positions i1 to iz of the edge of the liver, the trigger level TL is determined. FIG. 15 is a diagram illustrating an example of the trigger level TL. The trigger level TL expresses the reference position of the edge of the liver at the time of executing a data acquisition sequence DAQ (refer to FIG. 16) in the main scan B which will be described later. The trigger level TL can be set, for example, in an intermediate value between the maximum value and the minimum value of the position of the edge of the liver. How the trigger level TL is used at the time of executing the main scan B will be described later. After executing the pre-scan A, the main scan B is executed.

FIG. 16 is an explanatory diagram of the main scan B. In the main scan B, the navigator sequence NAV and the data acquisition sequence DAQ for acquiring data of the liver are executed.

Also in the main scan B, the navigator system NAV is executed according to the flow illustrated in FIG. 12 to detect the position of the edge of the liver.

In such a manner, changes with time of the position of the edge of the liver are monitored. When the position of the edge of the liver moves from the upper side of the trigger level TL to the lower side, the data acquisition sequence DAQ is executed.

Similarly, the navigator sequence NAV and the data acquisition sequence DAQ are repeatedly executed, and the main scan B is finished. On the basis of the data acquired by the main scan B, an image of the liver is reconstructed, and the imaging of the subject is finished.

In the embodiment, the sum Sₗᵢᵥₑᵣ of signal intensities in the liver region and the sum S_{lung} of signal intensities in the lung region are compared, and a channel when Sₗᵢᵥₑᵣ>S_{lung} is satisfied is selected as a channel used to detect the position of the edge of the liver. Therefore, a channel when Sₗᵢᵥₑᵣ≦S_{lung} is satisfied is not selected as a channel used to detect the position of the edge of the liver, so that the precision of detection of the position of the edge of the liver can be increased.

In the embodiment, the sum Sₗᵢᵥₑᵣ of signal intensities in the liver region and the sum S_{lung} of signal intensities in the lung region are calculated. However, if a feature amount of the signal intensities in the liver region and a feature amount of the signal intensities in the lung region can be obtained, values different from the sums Sₗᵢᵥₑᵣ and S_{lung} of the signal intensities may be calculated. For example, an average value S1 of the signal intensities in the liver region may be calculated in place of the sum Sₗᵢᵥₑᵣ of signal intensities in the liver region, and an average S2 of the signal intensities in the lung region may be calculated in place of the sum S_{lung} of the signal intensities of the lung region. In the case of calculating the average values S1 and S2 of the signal intensities, it is sufficient to select a channel when S1>S2 is satisfied as a channel used to detect the position of the edge of the liver. In this case, a channel when S1≦S2 is satisfied is not selected as a channel used to detect the position of the edge of the liver, so that the precision of detection of the position of the edge of the liver can be increased.

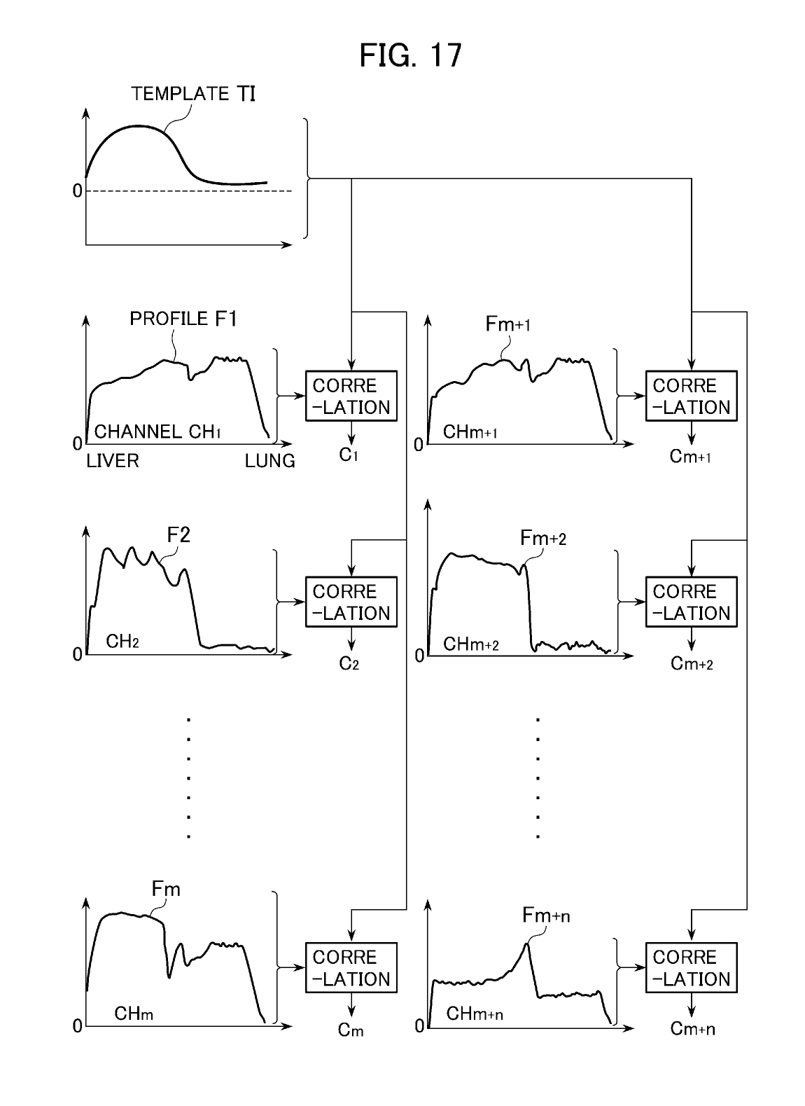

In the embodiment, by comparing the sum Sₗᵢᵥₑᵣ of signal intensities in the liver region and the sum S_{lung} of signal intensities in the lung region, a channel is selected. On the other hand, it is also considered to prepare a template expressing an ideal signal intensity of each position in the navigator region, obtain a correlation coefficient between the template and each profile, and select a channel when the correlation coefficient is large (refer to FIG. 17).

FIG. 17 is an explanatory diagram of an example of a method of selecting a channel by using a template TI.

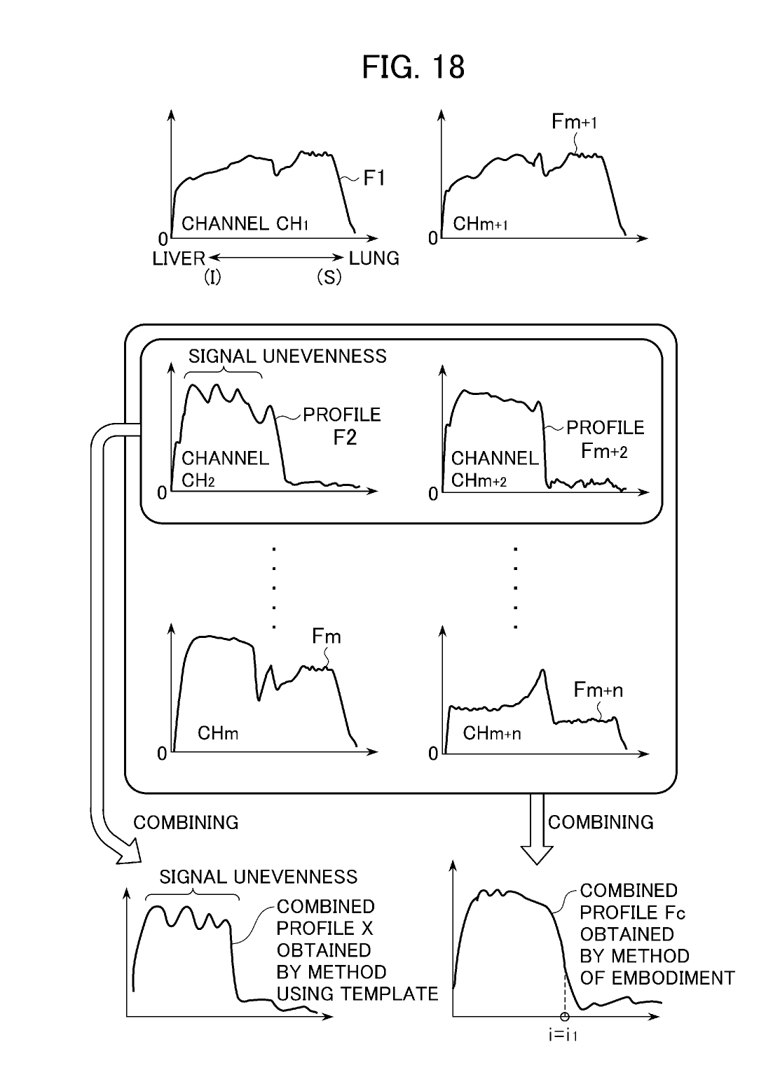

In FIG. 17, the template TI is illustrated. The template TI is data expressing ideal signal intensity in each position in the navigator region. In the method using the template TI, correlation coefficients C₁ to Cₘ₊ₙ between the template TI and the profiles F₁ to Fₘ₊ₙ are obtained, and a channel in which the correlation coefficient is large is selected from the channels CH1 to CHₘ₊ₙ. Therefore, a channel in which the correlation coefficient is small is not selected, so that the precision of detecting the position of the edge of the liver can be increased. In this method, however, it is considered to select only a channel in which the correlation coefficient is as high as possible. The number of channels selected is small and, generally, it is set to select only the channel in which the correlation coefficient is the largest and the channel in which the correlation coefficient is the second largest (that is, two channels). For example, when it is assumed that, in FIG. 17, the correlation coefficient C₂ of the channel CH₂ is the largest and the correlation coefficient Cₘ₊₂ of the channel CHₘ₊₂ is the second largest in the correlation coefficients C₁ to Cₘ₊ₙ, only the two channels CH₂ and CHₘ₊₂ are selected. Therefore, in the method using the template TI, a profile F₂ of the channel CH₂ and a profile Fₘ₊₂ of the channel CHₘ₊₂ are combined (refer to FIG. 18).

FIG. 18 is a diagram schematically illustrating a composite profile X obtained by using the method of using the template TI. In FIG. 18, the composite profile Fc obtained by the method of the embodiment is also illustrated.

There is a case that signal unevenness appears in the liver region of the profile depending on imaging parameters or the like. FIG. 18 illustrates an example when signal unevenness appears in the liver region in the profile F₂. Generally, the signal unevenness in the liver region tends to appear as the number of channels of the coil becomes larger. In the case where such signal unevenness appears in the profile F₂, only by combining the profiles F₂ and Fₘ₊₂, signal unevenness in the profile F₂ cannot be sufficiently reduced, and signal unevenness appears also in the region of the liver in the composite profile X. When signal unevenness appears in the composite profile X, it causes deterioration in the precision of detecting the position of the edge of the liver.

On the other hand, in the embodiment, the template TI is not used. The sum Sₗᵢᵥₑᵣ of signal intensities in the liver region and the sum S_{lung} of signal intensities in the lung region are compared, and a channel where Sₗᵢᵥₑᵣ>S_{lung} is satisfied is selected as a channel used to detect the position of the edge of the liver. Therefore, the channel where Sₗᵢᵥₑᵣ>S_{lung} is satisfied is selected as a channel used to detect the position of the edge of the liver regardless of the correlation coefficient. Consequently, in the method of the embodiment, as compared with the method using the template, larger number of channels can be selected as channels used at the time of detecting the position of the edge of the liver. Referring to FIG. 18, it is understood that, in the method using the template, only the channels CH₂ and CHₘ₊₂ (that is, two channels) are selected and, in the method of the embodiment, the channels CH₂ to CHₘ and CHₘ₊₂ to CHₘ₊ₙ are selected. Therefore, in the method of the embodiment, larger number of profiles are combined than that in the method using the template, so that the composite profile Fc in which the influence of the signal unevenness of the channel CH₂ is sufficiently reduced can be obtained, and the precision of detecting the position of the edge of the liver can be improved.

In the embodiment, the navigator region Rₙₐᵥ is set so as to include the liver and the lung. As long as a body site which is moves is included, the navigator region Rₙₐᵥ may include parts different from the liver or lung. For example, the navigator region Rₙₐᵥ may be set so as to include the liver and the heart.

In the embodiment, on the basis of a navigator signal obtained by the navigator sequence NAV at the time t1 of the pre-scan A, a channel used to detect the position of the edge of the liver is selected from the channels CH₁ to CHₘ₊ₙ. It is also possible to execute the navigator sequence NAV for selecting a channel twice or more and select a channel on the basis of navigator signals obtained by the navigator sequences NAV.

In the embodiment, the position of the edge of the liver is detected according to the flow of FIG. 6 at time t1 and the position of the edge of the liver is detected according to the flow of FIG. 12 at time t2 and after that. However, also at time t2 and after that, the position of the edge of the liver may be detected according to the flow of FIG. 6.

In the embodiment, the example of acquiring data by triggering has been described. The present invention, however, is not limited to triggering but can be applied to any imaging as long as a navigator signal has to be received by a coil having a plurality of channels.

1. A magnetic resonance apparatus for obtaining a navigator signal generated from a navigator region including a first body site which moves and a second body site which moves by using a coil having a plurality of channels, comprising:
scan means for executing a first navigator sequence for obtaining a first navigator signal generated from the navigator region;
profile generating means for generating a first profile expressing relation between each position in the navigator region and signal intensity, for each of the channels, on the basis of the first navigator signal received by each of the plurality of channels;
means for obtaining a first region corresponding to the first body site in the first profile and a second region corresponding to the second body site in the first profile; and
selecting means for selecting a channel used to obtain the position of the first body site from the plurality of channels on the basis of a feature amount of the signal intensity in the first region and a feature amount of the signal intensity in the second region.

2. The magnetic resonance apparatus according to claim 1, wherein:
the feature amount of the signal intensity in the first region is a sum of the signal intensities in the first region or an average value of the signal intensities in the first region, and
the feature amount of the signal intensity in the second region is a sum of the signal intensities in the second region or an average value of the signal intensities in the second region.

3. The magnetic resonance apparatus according to claim 1, wherein the means for obtaining the first region and the second region obtains position of a border between the first body site and the second body site and, using the position of the border as a reference, obtains the first region and the second region.

4. The magnetic resonance apparatus according to claim 3, wherein the means for obtaining the first region and the second region obtains the position of the border on the basis of the signal intensity of the first profile.

5. The magnetic resonance apparatus according to claim 3, wherein the means for obtaining the first region and the second region obtains, as the position of the border, an intermediate position of the navigator region.

6. The magnetic resonance apparatus according to claim 1, wherein:
the scan means executes a second navigator sequence for obtaining a second navigator signal generated from the navigator region, and
the profile generating means generates a second profile expressing the relation between each of positions in the navigator region and the signal intensity on the basis of the second navigator signal received by the channel selected by the selecting means.

7. The magnetic resonance apparatus according to claim 6, further comprising means for obtaining the position of the first body site on the basis of the second profile.

8. The magnetic resonance apparatus according to claim 7, wherein:
the selecting means selects two or more channels from the plurality of channels,
the profile generating means generates the second profile for each selected channel on the basis of the second navigator signal received by the two or more channels selected by the selecting means, and
the means obtaining the position of the first body site combines the second profiles obtained for the selected channels and obtains the position of the first body site on the basis of a profile obtained by the combining.

9. The magnetic resonance apparatus according to claim 7, wherein the means for obtaining the position of the first body site combines the first profiles obtained for the selected channels and obtains the position of the first body site on the basis of a profile obtained by the combining.

10. The magnetic resonance apparatus according to claim 1, wherein:
the first body site includes an edge of a liver, the second body site includes a part of a lung, and
the position of the first body site is the position of the edge of the liver.

11. A program applied to a magnetic resonance apparatus executing a first navigator sequence for obtaining a first navigator signal generated from a navigator region including a first body site which moves and a second body site which moves by using a coil having a plurality of channels, the program for making a computer execute:
a profile generating process generating a first profile expressing relation between each position in the navigator region and signal intensity for each of the channels on the basis of the first navigator signal received by each of the plurality of channels;
a process obtaining a first region corresponding to the first body site in the first profile and a second region corresponding to the second body site in the first profile; and
a selecting process selecting a channel used to obtain the position of the first body site from the plurality of channels on the basis of a feature amount of the signal intensity in the first region and a feature amount of the signal intensity in the second region.
